(19) United States
(12) Patent Application Publication
(10) Pub. No.: US 2023/0310305 A1
(43) Pub. Date: Oct. 5, 2023

(54) Topical Varins for Hair Growth

(71) Applicant: Varin Venture Group LLC, Greenwood Village, CO (US)

(72) Inventors: Gregory Smith, St. Pete Beach, FL (US); William A. Spilo, Greenwood Village, CO (US)

(21) Appl. No.: 18/130,600

(22) Filed: Apr. 4, 2023

Related U.S. Application Data
(60) Provisional application No. 63/327,332, filed on Apr. 4, 2022.

(51) Int. Cl. A61K 8/9789; A61K 8/34; A61K 8/63; A61K 8/92; A61Q 7/00

(52) U.S. Cl. CPC A61K 8/9789; A61K 8/34; A61K 8/63; A61K 8/922; A61Q 7/00

(57) ABSTRACT

Once daily topical application of a high in CBD, THCV and CBDV cannabis extract formulation, averaging about 33 mg per day for six months results in increased hair growth in both male and female subjects. Males showed an average hair count increase of 246% (15.5 new hairs per cm²) and females showed an increase of 127% (also 15.5 new hairs per cm²).

### CROSS-REFERENCE TO PRIOR APPLICATIONS

The present application is based on and claims the priority and benefit of U.S. Provisional Patent Application Ser. No. 63/327,332, filed on 4 Apr. 2022.

### U.S. GOVERNMENT SUPPORT

N/A

### BACKGROUND OF THE INVENTION

### Area of the Art

The present application is in the area of topical compositions for stimulating hair growth and more specifically relates to the use of a class of cannabinoids known as varins for this purpose.

### DESCRIPTION OF THE BACKGROUND ART

Human beings are one of the few primates that have head hair that shows essentially indeterminate growth (growth of individual hair strands lasting for several years). (This is also true of the facial hair (beard) exhibited by human males.) Most mammals have body hair (“fur”) that exhibits determinate growth (growth of individual strands lasting only days or weeks) so that hairs are all of a uniform length. This is true also of the sparse body hair that most humans exhibit. It is not clear why human head hair shows an indeterminate growth pattern. In any case human head hair seems to cause a lot of problems. Both males and females may spend a lot of time styling and arranging their hair. Hair is often considered to affect an individual's attractiveness. Therefore, loss of hair is often distressing to an individual.

Androgenetic alopecia (AGA) is a common condition that occurs in both males and females and increases in prevalence with age. It is by far the most common cause of baldness. It generally starts in the third and fourth decades of life and significantly increases in prevalence in women after menopause.^{2, 3} Is it estimated that 50% of Caucasian males and 19% of Caucasian females are affected by age 50, and there is a lower prevalence and severity of the condition in Asian and black males.⁴ AGA may adversely impact a person both psychologically and socially, especially in females⁵ The condition is characterized by follicular miniaturization in a specific pattern due to the effects of systemic androgens and genetic factors.⁶

In the male pattern phenotype, the hairline regresses at the bitemporal regions and at the vertex, and in the female pattern, there is diffuse thinning with preservation of the frontal hairline; however, the pathogenesis is the same.^{5, 7} AGA develops due to a disturbance in the cyclic transformation of hair follicles from active hair shaft growth and pigment production (anagen phase) to apoptosis-driven (cell death) hair follicle involution (catagen phase).²

The pathology of hair loss, especially female AGA, is complex with many gaps in our understanding of pathophysiology. In normal head hair growth, the hair follicles cycle through distinct phases.⁹ Growth, known as anagen phase, can last up to six years. Ninety percent of the follicles are usually in this phase. In this phase the hair shaft extends and thickens due to matrix cell activity. This is followed for the catagen or resting phase. Only about one percent of follicles are usually in this phase. In this phase, the follicle regresses. This is followed by a period of quiescence known as the telogen phase. About nine percent of follicles are usually in this phase. The last phase is call the exogen phase with the release of the hair shaft. The proportion of follicles in anagen phase declines with age.^{10, 11}

The signals that cause the transition between the various phases of the hair follicle growth are not well understood. There is complex signaling cycles between the root sheath and the dermal papillae, involving several protein families including fibroblast growth factors and bone morphogenic proteins; sonic hedgehog and Wnt signaling are involved.¹¹

In males, androgens are usually involved. It is known that in many males, dihydrotestosterone (DHT), synthesized from testosterone by 5a reductases, is a major player.¹² The intracellular signaling cascade after androgen receptor binding by DHT is poorly understood. In females, there is less evidence regarding the involvement of androgens. The higher prevalence of AGA in post-menopausal females, however, does indicate some sort of association with hormone levels.¹³

Consequently, there is considerable economic activity dealing with hair loss. The oldest means of dealing with hair loss is cosmetic including a variety of wigs, toupees, colorants, hair building fibers, etc. However, many individuals seek a more permanent solution to hair loss. The most successful medical answer is probably hair transplantation in which hair follicles are transplanted from a region of the head that has not experienced hair loss to a region where hair has been lost. Unfortunately, hair transplantation is expensive, can be painful, and is limited by the availability of hair follicles from reasons that have not experienced hair loss.

The potential drawbacks to hair transplantation has led to a search for topical compositions and drugs that can counteract hair loss. There is a long history of various herbal compositions that supposedly retard or reverse hair loss. Unfortunately, there is little reliable data to support the efficacy of such compositions. Perhaps the first topical treatment for hair loss that was supported by scientific data was minoxidil. This compound was originally developed as a blood pressure lowering agent that worked by promoting vascular dilation. Patients taking the drug showed enhanced hair growth, and this effect was shown to extend to topical applications of minoxidil (allowing targeted hair growth enhancement).

Only two medications, topical minoxidil and oral finasteride, are FDA approved for the treatment of AGA. Then can be used alone or in combination for synergistic effects.¹⁴ The FDA has approved no new medications in over 15 years. Finasteride, a 5a reductase inhibitor, blocks the conversion of testosterone to DHT¹⁵ has proven useful in the treatment of AGA³. However, finasteride is associated with several side effects, including sexual dysfunction that in some cases persists after ceasing therapy.¹⁶ Despite a small number of studies showing efficacy, the use of finasteride in women remains controversial, and only off-label, uncontrolled studies and anecdotal evidence have reported positive results¹⁷. Finasteride treatment in pre-menopausal women is also problematic, requiring concomitant contraceptive treatment due to the teratogenic nature of the compound.¹⁷

Topical minoxidil is the only known treatment that is effective in both males and females; however, the mechanism of action is unknown.¹⁸ Minoxidil is generally well tolerated but is also associated with several side effects, including an initial increase in hair shedding and exacerbation of hair loss following withdrawal from treatment.¹⁹

Unfortunately, these medications offer limited results.²⁰⁻²² Recently, the combination of topical minoxidil and topical finasteride has shown more promising results.^{14, 23} Surgical hair transplantation is the only current successful permanent option. Several other medical options, including antiandrogens such as spironolactone, oral contraceptives, cyproterone, flutamide, dutasteride, prostaglandin analogs, and ketoconazole are reported to be beneficial. However, these treatments can be associated with significant adverse effects and are expensive.²² Laser and light therapies have also become popular despite the lack of documented profound benefit.²⁴

A phase III clinical trial of men with AGA was conducted in 2021. The study used an investigational new topical drug called SM04554 which works by modulating the Wnt pathway that is postulated to initiate and maintain the anagen phase of the hair cycle.²⁵ Wnt signaling also causes dermal progenitor cells to differentiate into new hair follicles. It is interesting to note that CBD has also been shown to increase Wnt signaling.²⁶ To date, however, there is little basic science or clinical research on CBD and Wnt signaling.

A 2014 study investigated hair growth in mice using 3% peppermint oil compared to 3% minoxidil and jojoba oil. The results showed that peppermint oil (40% menthol) “showed the most prominent hair growth effects; a significant increase in dermal thickness, follicle number, and follicle depth.” The active ingredient in peppermint oil appears to be menthol, and peppermint oil is normally 40-50% menthol. The researchers suggested that peppermint oil induces a rapid anagen stage.¹

Recently, with the increasing acceptance of Cannabis sativa-based therapies, hemp extract has come under consideration as a possible, effective, safe, inexpensive non-prescription, topical AGA therapy. A 2021 case study²⁷ of CBD-rich hemp extract demonstrated 93.5% average increase in hair regrowth. Hemp extract works through the ECS in the body and has novel effects on hair follicle elongation and hair matrix keratinocytes activated through ECS receptors in the hair follicle cells.²⁷ As such, the therapeutic effects of hemp extract should complement the physiologic effects of minoxidil, finasteride, and antiandrogen therapies.

THCV and CBDV are full CB1 antagonists, compared to CBD which is a partial CB1 antagonist.^{28, 29} Therefore, the therapeutic effects from CB1 blockade should be more marked with the addition of THCV and CBDV.^{28, 30 31} Until recently hemp extract with significant percentages of THCV and/or CBDV have been unavailable commercially.

The ECS was discovered in the 1990s. In essence, it is a system involved with maintaining cellular homeostasis in response to excess oxidative stress. It downregulates the damaging inflammatory response, and it upregulates regenerative processes. It is comprised of at several receptors, including cannabinoid receptor 1 and 2 (CB1 and CB2), vanilloid receptor-1 (TRPV1) and vanilloid receptor-4 (TRPV4). It has at least two messenger molecules known as the endocannabinoids, anandamide (AEA) and 2-arachidonylglycerol (2-AG).³² One of the many systems that the ECS is involved with is thermoregulation within the skin. There are a substantial number of CB1 and CB2 receptors on various cell types within the skin.³³ CB1 receptors are well expressed in hair follicle cells.³⁴

The hair follicle cycle (anagen, catagen, and telogen phases) is controlled by the TRPV1.³⁵ TRPV1 receptors are found on hair matrix keratinocytes. Mouse studies have shown that activation TRPV1 receptors promotes hair follicle regression (catagen) and hair matrix keratinocyte apoptosis (cell death) and retardation of hair shaft elongation.³⁵ Endocannabinoids, and cannabis-derived phytocannabinoids, such as tetrahydrocannabinol (THC) and CBD, message TRPV1 receptors. It is postulated that CBD has therapeutic effects via TRPV1 receptors by such excessive activation of the receptor that then become desensitized. ³⁶

THC is a CB1 receptor agonist, and it has been shown to dose-dependently inhibit hair shaft elongation, decrease proliferation of hair matrix keratinocytes, and induce intraepithelial apoptosis and premature hair follicle regression (catagen). These effects from THC were inhibited by a selective CB1 antagonist.^{34, 35}

The available research suggests that THC and other CB1 agonists can be used to manage unwanted hair growth, and likewise, CB1 antagonists, such as CBD, THCV and CBDV, can be used to promote hair growth.³⁴ CBD is a CB1 partial antagonist that probably produces its effects via negative allosteric modulation of the CB1 receptor.^{37, 38} Whereas, THCV and CBDV are full neutral CB1 receptor antagonists.²⁹

A recent study of human hair follicle cultured cells revealed that use of lower doses of CBD resulted in hair shaft elongation, probably via CB1 antagonism.³⁹ However, much higher doses resulted in premature entry into the catagen phase, probably via a different receptor, the vanilloid receptor-4 (TRPV4). Therefore, the dosing of the topical CBD needs to be controlled to obtain positive hair growth.

Over the past decade, CBD has been extensively researched for a myriad of therapeutic benefits.⁴⁰ CBD does not cause euphoria or addiction. It is safe, with a wide therapeutic window and few adverse effects. Topical application of CBD has not been associated with any significant adverse effects.^{33, 35} CBD in an oral form has been FDA approved for treatment of recalcitrant epilepsy. CBD in sublingual, oral, inhaled, and topical versions are relatively inexpensive and widely available as nutraceuticals. It is estimated that 14% of the U.S. population has tried CBD products.⁴¹

The cannabinoids such as CBD are fat-soluble and poorly absorbed through the epidermis, but properly formulated topical applications reach hair follicles where it is a CB1 antagonist and TRPV1 and TRPV4 agonist.³⁹

THCV and CBDV, known together as the ‘varins,’ have not been as extensively researched because of the dearth of available hemp (low-THC Cannabis sativa) extract containing any significant amounts of these two cannabinoids. There has been considerable research of ingested varins derived from marijuana (high-THC Cannabis sativa) for the treatment of epilepsy⁴², obesity⁴³ and diabetes mellitus.⁴³

### SUMMARY OF THE INVENTION

A study was conducted on subjects with androgenetic alopecia (AGA), to determine if daily topical application of a formulation with high CBD, THCV and CBDV concentrations would result in improved hair regrowth in the area of the scalp most affected by AGA. The study included 31 (15 men and 16 women, 27 Caucasian, 2 Asian and 1 mixed race) subjects. Participant ages ranged from 31 to 65 for the females and 39 to 64 for the males. The subjects gave their written informed consent for the six-month trial. The study adhered to the Helsinki guidelines and was institutionally approved. None of the subjects were currently using minoxidil or finasteride. No other hair loss treatments were used during the six months of the research study.

They used a once daily topical application of a high in CBD, THCV and CBDV cannabis extract formulation, averaging about 33 mg per day for six months. A hair count of the area showing the most significant alopecia was carried out before treatment was started and again after six months of treatment. To facilitate consistent hair count analysis, a permanent tattoo mark was placed at the point showing maximum hair loss on the scalp.

The subjects were photographed in a standard manner before and after the study. The photographs were compared for improvements in ‘scalp coverage’ by an independent physician. The qualitative scale included “none”, “mild”, “moderate”, “extensive” improvement of scalp coverage.

A hair count of the greatest area of alopecia was carried out before treatment was started and again after six months of treatment. To facilitate consistent hair count analysis a permanent black tattoo dot was placed at the point of maximum hair loss on the scalp. The non-vellus hairs within the 1 cm² around the tattoo were pulled through the opening of a one-centimeter mold with a surgical skin hook and a hair count taken using a Bodelin ProScope with 50× magnification.

For all males, the baseline hair count was 6.13/cm² and at six months, it was 21.20/cm² (one-tailed paired t-test p<0.00001). This represented an average increase of 246% or 15.50 additional hairs in the one square centimeter mold. For all females, the baseline hair count was 12.69/cm² and at six months, it was 28.75/cm2 (one-tailed paired t-test p<0.00001). This represented an average increase of 127% or 15.50 additional hairs in the one square centimeter mold. For all adults the baseline hair count was 9.50/cm² and it increased after six months to 25.00 (one-tailed paired t-test p<0.00001). This represented an average increase of 164% or 15.50 additional hairs in the one square centimeter mold. All subjects showed some increase in hair count. The increase ranged from 31.25% in a female (16 to 21 hairs/cm²) to 2000% in a male (1 to 21 hairs/cm²). In general, the increased hair counts were associated with a cosmetically pleasing result.

This case study showed that topical hemp extract high in THCV, CBDV, CBD, menthol and peppermint oil is associated with significant hair regrowth in both men and women with AGA. This topical was superior to high-CBD hemp extract alone.²⁷ In general, men did better than women. On average, there was a 164% (p<0.00001) increase in non-vellus hair after six months of once-daily use. All subjects had some regrowth and cosmetic benefits.

Although the exact mechanism of therapeutic effects is not currently known, CBD is most likely functioning as a CB1 receptor antagonist, via negative allosteric effects, and potentially also via Wnt messaging. THCV and CBDV are acting as full CB1 neutral antagonists and via TRPV1 agonism. The menthol and peppermint (40% menthol) are most likely acting by promoting the rapid onset of anagen phase¹. Although the test was carried out on head hair, the formulation is effective on any region supporting non-vellus hair such as the beard.

The safety of topically applied cannabis extract has been previously well documented. Once again, there are no reported significant adverse effects for six-month topical application of cannabis extract.^{25, 26}

Topical cannabis formulation results in superior results to finasteride and 5% minoxidil once daily foam. Since this extract works through novel mechanisms entirely different from finasteride and minoxidil, it can be used in conjunction with these current drugs and would be expected to have synergistic effects.

### DESCRIPTION OF THE FIGURES

FIG. 1 is a photograph of the scalp of a male participant at the beginning of the study (showing 2 hairs/cm²);

FIG. 2 is a photograph of the scalp of a male participant of FIG. 1 at the end of the study (showing 19 hairs/cm²);

FIG. 3 is a photograph of the scalp of a female participant at the beginning of the study (showing 16 hairs/cm²); and

FIG. 4 is a photograph of the scalp of a female participant of FIG. 3 at the end of the study (showing 37 hairs/cm²).

### DETAILED DESCRIPTION OF THE INVENTION

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein specifically to provide an improved hair growth promoting treatment based on a topical treatment high in cannabis-derived CBD, THCV and CBDV

Study Design. The study was a case series of adults presenting to a “Hair and Scalp” center in Clearwater, Florida. Adult subjects, who were not currently using minoxidil or finasteride, were offered the opportunity to receive the hemp-based formulation free of charge. Thirty-one subjects (15 males, 16 females, 27 Caucasian, 2 Asian and 1 Mixed race) had AGA with Norwood-Hamilton Classification score of 3V or higher. The predefined endpoints were hair counts obtained in a defined, representative area of scalp hair loss, and investigator clinical assessment of hair growth.

Participant ages ranged from 31 to 65 for the females and 39 to 64 for the males. The subjects gave their written informed consent for the six-month trial. The study adhered to the Helsinki guidelines and was institutionally approved. None of the subjects were currently using minoxidil or finasteride. No other hair loss treatments were used during the six months of the research.

The subjects were given a 60 ml (nominally two ounce) dispenser of product and advised to apply a thin layer once each morning to the areas of baldness. The dispenser contained 30 ml of formulation plus 30 ml of HFA 134a (1,1,1,2-tetrafluoroethane) propellant. It will be appreciated that each gram dispensed would contain about 0.5 g HFA and about 0.5 g formulation. However, the HFA evaporates essentially instantly leaving 0.5 g of formulation. The formulation was composed of a whole plant cannabis extract (CBD 60.00%, CBDV 12.63%, THCV 3.71%, delta 9 THC 0.18%, cannabigerol (CBG) 0.86% and cannabinol (CBN) 0.05% as independently analyzed by ACS Laboratory, Sun City Center, Florida). Each one ounce (approximately 30 g) of the formulation contained active ingredients of 1 g of the cannabis extract, 2 g saw palmetto seed extract, 1 g of menthol, 0.2 g of peppermint oil infused into a vehicle of 2.5 g of ethanol, 0.3 g pulegone, 0.6 g of Emu oil, 1 g of dimethicone and 21.4 g isolanolin. The one-ounce foam spray lasted approximately one month on average. This is an average daily dose of approximately 1 g of the formulation containing approximately 33 mg of the cannabis extract. The subjects were advised that they could use blow dryers, conditioners, and shampoos. The formulation was replaced as needed throughout the six-month trial.

A hair count of the greatest area of alopecia was carried out before treatment was started and again after six months of treatment. To facilitate consistent hair count analysis a permanent black tattoo dot was placed at the point of maximum hair loss on the scalp. The non-vellus hairs within the 1 cm² around the tattoo were pulled through the opening of a one-centimeter mold with a surgical skin hook and a hair count taken using a Bodelin ProScope with 50× magnification.

Results. For all males, the baseline hair count was 6.13/cm² and at six months, it was 21.20/cm² (one-tailed paired t-test p<0.00001). This represented an average increase of 246% or 15.50 additional hairs in the one square centimeter mold. For all females, the baseline hair count was 12.69/cm² and at six months, it was 28.75/cm2 (one-tailed paired t-test p<0.00001). This represented an average increase of 127% or 15.50 additional hairs in the one square centimeter mold. For all adults the baseline hair count was 9.50/cm² and it increased after six months to 25.00 (one-tailed paired t-test p<0.00001). This represented an average increase of 164% or 15.50 additional hairs in the one square centimeter mold. All subjects showed some increase in hair count. The increase ranged from 31.25% in a female (16 to 21 hairs/cm²) to 2000% in a male (1 to 21 hairs/cm²). In general, the increased hair counts were associated with a cosmetically pleasing result.

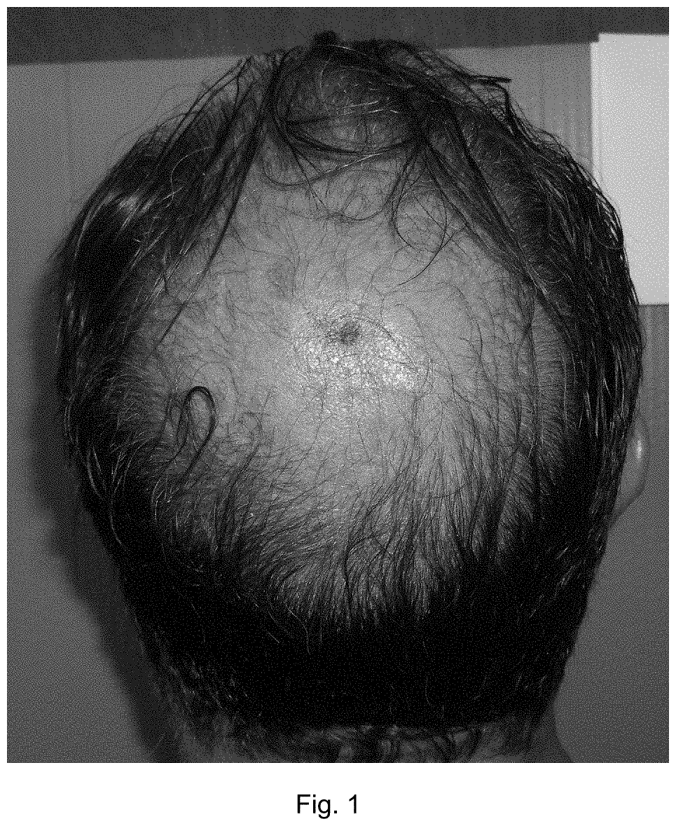

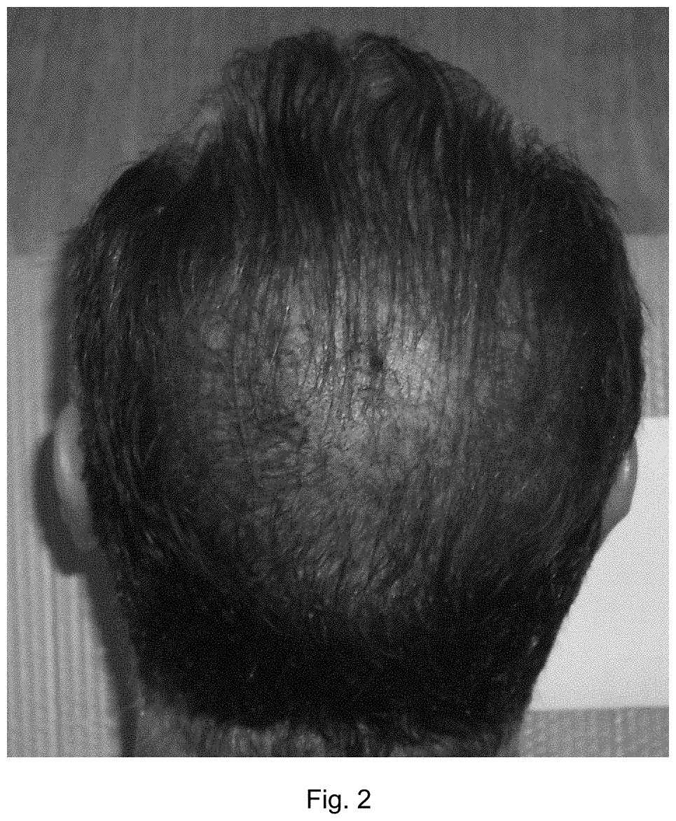

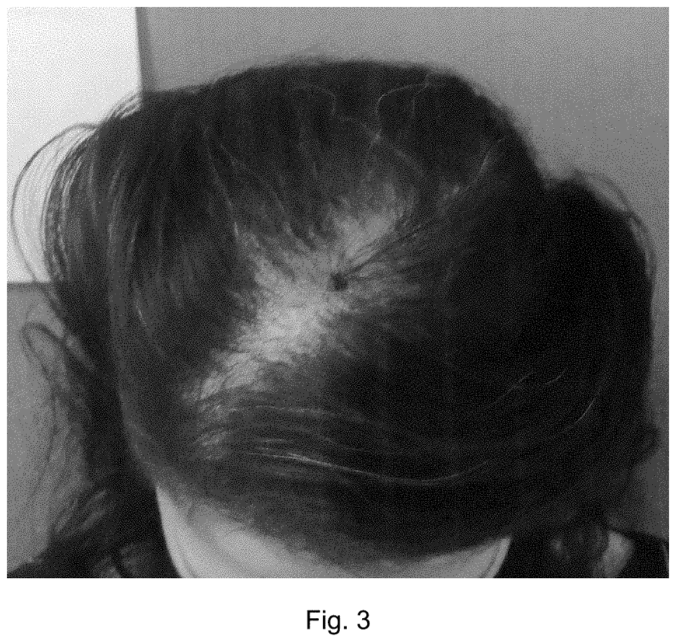

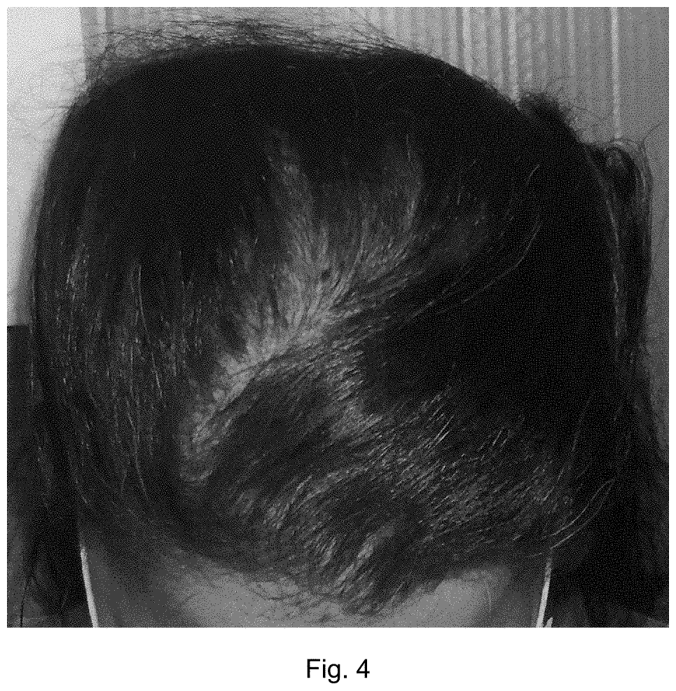

Independent physician review of the photographs revealed evidence of ‘mild’ to ‘extensive’ scalp coverage improvements for all subjects. FIG. 1 shows a typical male participant at the start of the study while FIG. 2 shows the same participant at the conclusion of the study. FIG. 3 shows a typical female participant at the start of the study while FIG. 4 shows the same participant at the conclusion of the study.

This study demonstrates that topical hemp extract high in THCV, CBDV, CBD, menthol and peppermint oil is associated with significant hair regrowth in both men and women with AGA. This topical was superior to high-CBD hemp extract alone.²⁷ In general, men did better than women. On average, there was a 164% (p<0.00001) increase in non-vellus hairs after six months of once-daily use. All subjects had some regrowth and cosmetic benefits.

The inventive topical cannabis formulation results in superior results to finasteride and 5% minoxidil once daily foam. Since this extract works through novel mechanisms entirely different from finasteride and minoxidil, it can be used in conjunction with these current drugs and is expected to have synergistic effects.

The following claims are to be understood to include what is specifically illustrated and described above, what is conceptually equivalent, what can be obviously substituted. Those skilled in the art will appreciate that various adaptations and modifications of the just-described preferred embodiment can be configured without departing from the scope of the invention. The illustrated embodiment has been set forth only for the purposes of example and that should not be taken as limiting the invention. Therefore, it is to be understood that, within the scope of the appended claims, the invention may be practiced other than as specifically described herein.

### REFERENCES

1. J Y O, M A P, Y C K. Peppermint Oil Promotes Hair Growth without Toxic Signs. Toxicological research. 214 December 2014; 30(4)doi:10.5487/TR.2014.30.4.297
2. Cranwell W, Sinclair R. Male Androgenetic Alopecia. Text. 2016/02/29 2016; doi:https://www.ncbi.nlm.nih.gov/books/NBK278957/
3. J M M, M C P, M M, H N C, G G. Efficacy and safety of finasteride therapy for androgenetic alopecia: a systematic review. Archives of dermatology. 210 October 2010; 146(10)doi:10.1001/archdermatol.2010.256
4. Krupa Shankar D, Chakravarthi M, Shilpakar R. Male Androgenetic Alopecia: Population-Based Study in 1,005 Subjects. Int J Trichology. 2009; 1(2):131-3. doi:10.4103/0974-7753.58556
5. Levy L L, Emer J J. Female pattern alopecia: current perspectives. Int J Womens Health. Aug. 29 2013; 5:541-56. doi:10.2147/ijwh.s49337
6. Salman K E, Altunay I K, Kucukunal N A, Cerman A A. Frequency, severity and related factors of androgenetic alopecia in dermatology outpatient clinic: hospital-based cross-sectional study in Turkey*. An Bras Dermatol. January-February 2017; 92(1):35-40. doi:10.1590/abd1806-4841.20175241
7. L L L, J J E. Female pattern alopecia: current perspectives. International journal of women's health. Aug. 29, 2013 2013; 5doi:10.2147/IJWH.S49337
8. T F C. The psychological effects of androgenetic alopecia in men. Journal of the American Academy of Dermatology. 1992 June 1992; 26(6)doi:10.1016/0190-9622(92)70134-2
9. K S S. The molecular and structural biology of hair: introduction. Annals of the New York Academy of Sciences. Dec. 26, 1991 1991; 642doi:10.1111/j.1749-6632.1991.tb24375.x
10. D A W. Male pattern hair loss: current understanding. International journal of dermatology. 1998 August 1998; 37(8)doi:10.1046/j.1365-4362.1998.00542.x
11. M C, G L, C H, J F G. Ageing and hair cycles. The British journal of dermatology. 1995 January 1995; 132(1)doi:10.1111/j.1365-2133.1995.tb08630.x
12. K D K. Androgens and alopecia. Molecular and cellular endocrinology. Dec. 30, 2002 2002; 198(1-2)doi:10.1016/s0303-7207(02)00372-6
13. A G M. Hair through the female life cycle. The British journal of dermatology. 211 December 2011; 165 Suppl 3doi:10.1111/j.1365-2133.2011.10628.x
14. L C, J Z, L W, H W, B C. The Efficacy and Safety of Finasteride Combined with Topical Minoxidil for Androgenetic Alopecia: A Systematic Review and Meta-analysis. Aesthetic plastic surgery. 220 June 2020; 44(3)doi:10.1007/s00266-020-01621-5
15. L D, M H, V F, et al. The effects of finasteride on scalp skin and serum androgen levels in men with androgenetic alopecia. Journal of the American Academy of Dermatology. 1999 October 1999; 41(4)
16. M S I, S K. Persistent sexual side effects of finasteride for male pattern hair loss. The journal of sexual medicine. 211 June 2011; 8(6)doi:10.1111/j.1743-6109.2011.02255.x
17. S M S, J L S. Finasteride treatment of hair loss in women. The Annals of pharmacotherapy. 210 June 2010; 44(6)doi:10.1345/aph.1M591
18. A G M, J R. Minoxidil: mechanisms of action on hair growth. The British journal of dermatology. 24 Feb. 2004; 150(2)doi:10.1111/j.1365-2133.2004.05785.x
19. M I K. Topical minoxidil: its use in treatment of male pattern baldness. Annals of plastic surgery. 1988 September 1988; 21(3)
20. A A, M G. The effectiveness of treatments for androgenetic alopecia: A systematic review and meta-analysis. Journal of the American Academy of Dermatology. 217 July 2017; 77(1)doi:10.1016/j.jaad.2017.02.054
21. A K G, A C. Topical Minoxidil: Systematic Review and Meta-Analysis of Its Efficacy in Androgenetic Alopecia. Skinmed. May-June 2015 2015; 13(3)
22. K Y, N M, B B, R S. A review of the treatment of male pattern hair loss. Expert opinion on pharmacotherapy. 220 April 2020; 21(5)doi:10.1080/14656566.2020.1721463
23. Suchonwanit P, Srisuwanwattana P, Chalermroj N, Khunkhet S. A randomized, double-blind controlled study of the efficacy and safety of topical solution of 0.25% finasteride admixed with 3% minoxidil vs. 3% minoxidil solution in the treatment of male androgenetic alopecia. J Eur Acad Dermatol Venereol. December 2018; 32(12):2257-2263. doi:10.1111/jdv.15171
24. Holmang S, Lele S M, Johansson S L. Squamous cell carcinoma of the renal pelvis and ureter: incidence, symptoms, treatment and outcome. J Urol. July 2007; 178(1):51-6. doi:10.1016/j.juro.2007.03.033
25. NCT03742518. A Study Evaluating the Efficacy and Safety of SM04554 Topical Solution in Male Subjects With Androgenetic Alopecia—Full Text View—ClinicalTrials.gov. CwwwClinicalTrialsgov. 2021;
26. Vallee A, Lecarpentier Y, Guillevin R, Vallee J N. Effects of cannabidiol interactions with Wnt/beta-catenin pathway and PPARgamma on oxidative stress and neuroinflammation in Alzheimer's disease. Acta Biochim Biophys Sin (Shanghai). Oct. 1 2017; 49(10):853-866. doi:10.1093/abbs/gmx073
27. Smith G, Satino J. Hair Regrowth with Cannabidiol (CBD)-rich Hemp Extract. Original Report. https://publicationscsciencesucfedu/cannabis/indexhp/Cannabis. 2021-03-25 2021; doi:https://publications.sciences.ucf.edu/cannabis/index.php/Cannabis/article/view/78
28. Laprairie R B, Bagher A M, Kelly M E M, Denovan-Wright E M. Cannabidiol is a negative allosteric modulator of the cannabinoid CB1 receptor. Br J Pharmacol. 2015:4790-805. vol. 20.
29. R G P. Inverse agonism and neutral antagonism at cannabinoid CB1 receptors. Life sciences. Feb. 4, 2005 2005; 76(12)doi:10.1016/j.lfs.2004.10.025
30. McPartland J M, Duncan M, Di Marzo V, Pertwee R G. Are cannabidiol and Δ9-tetrahydrocannabivarin negative modulators of the endocannabinoid system? A systematic review. Br J Pharmacol. February 2015; 172(3):737-53. doi:10.1111/bph.12944
31. Bolognini D. Pharmacological properties of the phytocannabinoids A9-tetrahydrocannabivarin and cannabidiol. Doctoral Thesis. 2010 2010; doi:http://hdl.handle.net/10277/269
32. Smith G L. Medical Cannabis: Basic Science and Clinical Applications. Aylesbury Press; 2016:225.
33. Tóth K F, Ádám D, Bird T, Oláh A. Cannabinoid Signaling in the Skin: Therapeutic Potential of the “C(ut)annabinoid” System. Molecules. 2019. vol. 5.
34. Telek Aea. Inhibition of human hair follicle growth by endo- and exocannabinoid. 2007;
35. Biro T, Toth B I, Hasko G, Paus R, Pacher P. The endocannabinoid system of the skin in health and disease: novel perspectives and therapeutic opportunities. Trends Pharmacol Sci. August 2009; 30(8):411-20. doi:10.1016/j.tips.2009.05.004
36. Muller C, Morales P, Reggio P H. Cannabinoid Ligands Targeting TRP Channels. Front Mol Neurosci. 2018; 11doi:10.3389/fnmol.2018.00487
37. Chung H, Fierro A, Pessoa-Mahana C D. Cannabidiol binding and negative allosteric modulation at the cannabinoid type 1 receptor in the presence of delta-9-tetrahydrocannabinol: An In Silico study. PLoS One. 2019; 14(7)doi:10.1371/journal.pone.0220025
38. Laprairie R B, Bagher A M, Kelly M E, Denovan-Wright E M. Cannabidiol is a negative allosteric modulator of the cannabinoid CB1 receptor. Br J Pharmacol. October 2015; 172(20):4790-805. doi:10.1111/bph.13250
39. Szabo ILea. 263 (-)-cannabidiol differentially influences hair growth I Request PDF. Journnal of Investigative Dermatology. 2017; doi:http://dx.doi.org/10.1016/j.jid.2017.07.261
40. Azer V B J, Charles A M et al. Collective View of CBD. Cowen Outperform. 2019;
41. Corroon J, Phillips J A. A Cross-Sectional Study of Cannabidiol Users. Cannabis Cannabinoid Res. 2018; 3(1):152-61. doi:10.1089/can.2018.0006
42. Gaston T E, Friedman D. Pharmacology of cannabinoids in the treatment of epilepsy. Epilepsy Behav. May 2017; 70(Pt B):313-318. doi:10.1016/j.yebeh.2016.11.016
43. A A, O A, A M, R P, A A, A S. A9-Tetrahydrocannabivarin (THCV): a commentary on potential therapeutic benefit for the management of obesity and diabetes. Journal of cannabis research. 01/31/2020 2020; 2(1)doi:10.1186/s42238-020-0016-7

What is claimed is:

1. A topical formulation for promoting hair growth comprising:
a cannabis extract containing both CBD, and
varins including at least about 5% of THCV and/or CBDV.

2. The topical formulation of claim 1, wherein the ratio of CBD to varins is between 3 and 4.

3. The topical formulation of claim 2 wherein the cannabis extract contains between about 55% and about 65% CBD.

4. The topical formulation of claim 1 further comprising a propellant.

5. The topical formulation of claim 1 further comprising peppermint oil.

6. The topical formulation of claim 1 further comprising menthol.

7. The topical formulation of claim 1 further comprising ethanol and isolanolin.

8. The topical formulation of claim 1, wherein the cannabis extract contains more than about 50% CBD and more than about 10% CBDV.

9. The topical formulation of claim 8, also containing more than about 2% THCV.

10. A method for promoting hair growth comprising the step of repeatedly applying a dose of a topical formulation to a region in need of hair growth, the formulation delivering at least about 3 mg of varins per dose.

11. A topical formulation for promoting hair growth wherein each 30 g of formulation contains active ingredients consisting essentially of:
1 g of a cannabis extract containing at least about 50% CBD, and at least about 1% THCV and at least about 5% CBDV;
about 2 g saw palmetto seed extract; and
at least about 1 g menthol.

12. A topical formulation for promoting hair growth comprising:
cannabinoids containing both CBD, and
varins including at least about 5% of THCV and/or CBDV.

13. The topical formulation of claim 12, wherein the ratio of CBD to varins is between 3 and 4.

14. The topical formulation of claim 13 wherein the cannabinoids include between about 55% and about 65% CBD.

15. The topical formulation of claim 12 further comprising peppermint oil.

16. The topical formulation of claim 12 further comprising menthol.

17. The topical formulation of claim 12, further comprising ethanol and isolanolin.

18. The topical formulation of claim 12, wherein the cannabinoids include more than about 50% CBD and more than about 10% CBDV.

19. The topical formulation of claim 8, also containing more than about 2% THCV.
